(19) United States
(12) Patent Application Publication
(10) Pub. No.: US 2023/0012831 A1
(43) Pub. Date: Jan. 19, 2023

(54) TWO-STEP PROCESS FOR CONVERTING LIQUEFIED WASTE PLASTICS INTO STEAM CRACKER FEED

(71) Applicant: Neste Oyj, Espoo (FI)

(72) Inventors: Marja TIITTA, Porvoo (FI); Pekka AALTO, Porvoo (FI); Andrea PÉREZ NEBREDA, Porvoo (FI); Ville PAASIKALLIO, Porvoo (FI); Antti OJALA, Porvoo (FI); John Jamieson, Porvoo (FI)

(73) Assignee: Neste Oyj, Espoo (FI)

(21) Appl. No.: 17/780,696

(22) PCT Filed: Nov. 27, 2020
(86) PCT No.: PCT/EP2020/083582

§ 371 Date: May 27, 2022

(30) Foreign Application Priority Data
Nov. 29, 2019 (FI) 20196034

(51) Int. Cl. C10G 1/10; C08J 11/14; C10G 17/02; C10G 45/08; C10G 1/00; C10G 9/36

(52) U.S. Cl. CPC C10G 1/10; C08J 11/14; C10G 17/02; C10G 45/08; C10G 1/002; C10G 9/36; C10G 2300/1003; C10G 2400/20

(57) ABSTRACT

The present disclosure relates to a method for upgrading liquefied waste plastics, the method including a step (A) of providing liquefied waste plastics (LWP) material, a step (B) including pre-treating the liquefied waste plastics material by contacting the liquefied waste plastics material with an aqueous medium having a pH of at least 7 at a temperature of 200° C. or more, followed by liquid-liquid separation, to produce a pre-treated liquefied waste plastics material, a step (C) including hydrotreating the pre-treated liquefied waste plastics material, optionally in combination with a co-feed, to obtain a hydrotreated material, and a step (D) of post-treating the hydrotreated material to obtain a steam cracker feed.

### TECHNICAL FIELD

The present invention relates to a two-step process for converting a waste plastics raw material, in particular liquefied waste plastics (LWP), into a steam cracker feed. Specifically, the present invention relates to a method for producing raw materials for chemical industry from plastic waste using a two-step process to provide a feed to be used in a steam cracking process.

### BACKGROUND OF THE INVENTION

The purification of liquefied waste plastics (LWP) to yield more valuable (pure) substances and the conversion of liquefied waste plastics (LWP) into more valuable material, such as low molecular olefins which can be used as raw material (e.g. as monomers) in chemical industry, have been studied for several years.

LWP is typically produced by hydrothermal liquefaction (HTL) or pyrolysis of waste plastics. Depending on the source of the waste plastics, LWP has variable levels of impurities. Typical impurity components are chlorine, nitrogen, sulphur and oxygen of which corrosive chlorine is particularly problematic for refinery/petrochemical processes. These impurities are also common in post-consumer waste plastics (recycled consumer plastics) that has been identified as the most potential large scale source for plastics waste. Similarly, bromine-containing impurities may be contained mainly in industry derived waste plastics (e.g. originating from flame retardants). Moreover, LWP produced by a pyrolysis process or hydrothermal liquefaction usually contains significant amounts of olefins and aromatics, which may lead to problems in some downstream processes, such as polymerisation (or coking) at elevated temperatures.

No matter whether the LWP is merely subjected to common refinery processing (e.g. including fractionation and optionally hydrotreatment) or is forwarded to a typical petrochemical conversion process (such as a cracking process), the LWP material needs to meet the impurity levels for these processes so as to avoid deterioration of the facility, such as corrosion of reactors or catalyst poisoning.

In addition to refining, chemical recycling of LWP back to plastic (or to monomers) is highly interesting option. This option has caught significant interest in the petrochemical industry during the last year. The interest has been further boosted by new waste directive and the EU plastic strategy that both set ambitious targets for the recycling of waste plastics.

It can thus be expected that chemical recycling will be an important method to recycle waste plastics back to plastics and chemicals in future. Liquefying of waste plastics and using it as feedstock for crackers (such as catalytic crackers, hydrocrackers or steam crackers) is also a promising method to recycle plastics because of the existing infrastructure. However, the potential of LWP as cracker feedstock depends on its quality and thus methods for purifying the LWP and/or modifying the cracking procedures have been proposed in order to handle the varying impurity contents of LWP.

WO 2018/10443 A1 discloses a steam cracking process comprising pre-treatment of a mainly paraffinic hydrocarbon feed, such as hydrowax, hydrotreated vacuum gas oil, pyrolysis oil from waste plastics, gasoil or slackwax. Pre-treatment is carried out using a solvent extraction so as to reduce fouling components, such as polycyclic aromatics and resins.

US 2016/0264874 A1 discloses a process for upgrading waste plastics, comprising a pyrolysis step, a hydroprocessing step, a polishing step and a stream cracking step in this order.

JP 2003-034794 A discloses a method for removing chlorine and nitrogen contaminants contained in waste plastic pyrolysis oil by contacting with a hot aqueous solution of alkaline metal compound or alkaline earth metal compound, followed by liquid-liquid separation.

Kawanishi, T., Shiratori, N., Wakao, H., Sugiyama, E., Ibe, H., Shioya, M., & Abe, T., “Upgrading of Light Thermal Cracking Oil Derived from Waste Plastics in Oil Refinery. Feedstock recycling of plastics.” Universitätsverlag Karlsruhe, Karlsruhe (2005), p. 43-50 discloses hydrotreating a blend of petroleum fractions and light thermal cracking oil from waste plastics to avoid fouling of a heat exchanger preceding the hydrotreater.

### SUMMARY OF INVENTION

The above prior art approaches employ complicated purification procedures, of which extraction techniques may result in significant amounts of contaminated extraction material, or provide a material which is still not fully suitable for steam cracking and leads to fouling and reduced service life of the steam cracker. There is still need for a more sustainable process allowing recycling large amounts of LWP while producing low amounts of waste products.

The present invention was made in view of the above-mentioned problems and it is an object of the present invention to provide an improved process for upgrading LWP, in particular a more sustainable process allowing recycling large amounts of LWP while producing low amounts of waste products and/or achieving improved service life of the steam cracker.

This problem of providing an improved process for upgrading LWP is solved by a method of claim 1, which comprises a step (A) of providing liquefied waste plastics (LWP) material, a step (B) comprising pre-treating the liquefied waste plastics material by contacting the liquefied waste plastics material with an aqueous medium having a pH of at least 7 at a temperature of 200° C. or more, followed by liquid-liquid separation, to produce a pre-treated liquefied waste plastics material, a step (C) comprising hydrotreating the pre-treated liquefied waste plastics material (optionally together with a co-feed) to obtain a hydrotreated material, and a step (D) of post-treating the hydrotreated material to obtain a steam cracker feed.

The method of the present invention makes use of the finding that a combination of reactive extraction with an aqueous solution of pH 7 or more at 200° C. or more and subsequent hydrotreatment can provide a material (steam cracker feed) which is ready to be fed to a steam cracker without the need for further purification or dilution.

In the present invention, steam cracking is employed because of its robustness regarding impurities, although steam crackers have typically strict specification for the chlorine content and the levels of several other impurities/components such as N, S, O, olefins and aromatics are controlled as well. The present inventors found that even LWP material which is pre-treated with conventional means may not always reach the required impurity restrictions and thus further purification would be required.

For example, hydrotreatment has been used in the prior art for removal of several impurities conventionally present in waste plastics, such as chlorine and sulphur contaminations. However, mere hydrotreatment requires large amounts of hydrogen and is thus less sustainable, in particular in view of the fact that most of the world's hydrogen production is still based on fossil sources.

Other approaches, such as WO 2018/10443 A1, employ pre-treatment using solvent extraction with an organic solvent or a water-based solvent. However, when employing organic solvents, large amounts of contaminated solvents are generated which require energy-consuming recovery procedures or which are used as low quality fuels, thus similarly failing to achieve a sustainable process. In any case, problematic impurities, such as aromatics, resins and olefins cannot be easily removed, which results in increased fouling.

The present inventors found that a combination of reactive extraction and hydrotreatment provides a steam cracker feed which has low impurity levels and does not cause excessive fouling. Specifically, the present inventors found that reactive extraction using an aqueous medium at pH 7 or more at 200° C. or more removes not only chlorine contaminants and to some degree nitrogen contaminants (both of which are undesired in steam cracker feeds) but furthermore can remove silicon-containing contaminants (such as organic silicon compounds and/or colloidal inorganic silicon material), thus enabling efficient hydrotreatment of pre-purified LWP material.

In the present invention, it is thus essential that the hydrotreatment step is preceded by the pre-treatment step so that silicon containing impurities are removed. Such Si impurities could otherwise result in catalyst poisoning of the hydrotreating catalyst. In the course of the pre-treatment step, sulphur impurities may be removed as well. Thus, in this case, it is an option to add a sulphur material (spiking) before hydrotreatment in order to ensure catalyst activity of the hydrotreating catalyst.

In brief, the present invention relates to one or more of the following items:

1. A method for upgrading liquefied waste plastics, the method comprising:
a step (A) of providing liquefied waste plastics (LWP) material,
a step (B) comprising pre-treating the liquefied waste plastics material by contacting the liquefied waste plastics material with an aqueous medium having a pH of at least 7 at a temperature of 200° C. or more, followed by liquid-liquid separation, to produce a pre-treated liquefied waste plastics material,
a step (C) comprising hydrotreating the pre-treated liquefied waste plastics material, optionally in combination with (or together with) a co-feed (hydrotreatment co-feed), to obtain a hydrotreated material, and
a step (D) of post-treating the hydrotreated material to obtain a steam cracker feed.

2. The method according to item 1, further comprising
a step (E) of subjecting the steam cracker feed to steam cracking.

3. The method according to item 1 or 2, wherein the pre-treated liquefied waste plastics material has a chlorine content of 5 wt.-ppm or more.

4. The method according to any one of the preceding items, wherein the pre-treated liquefied waste plastics material has a chlorine content of 10 wt.-ppm or more, 15 wt.-ppm or more, or 20 wt.-ppm or more.

5. The method according to any one of the preceding items, wherein the pre-treated liquefied waste plastics material has a chlorine content of 1000 wt.-ppm or less, 600 wt.-ppm or less, 400 wt.-ppm or less, 300 wt.-ppm or less, 200 wt.-ppm or less, 100 wt.-ppm or less, or 50 wt.-ppm or less.

6. The method according to any one of the preceding items, wherein the pre-treated liquefied waste plastics material has an olefins content of 10 wt.-% or more, 15 wt.-% or more, 20 wt.-% or more, 30 wt.-% or more, 40 wt.-% or more, or 50 wt.-% or more.

7. The method according to any one of the preceding items, wherein the pre-treated liquefied waste plastics material has an olefins content of 85 wt.-% or less, 80 wt.-% or less, 70 wt.-% or less, or 65 wt.-% or less.

8. The method according to any one of the preceding items, wherein the pre-treated liquefied waste plastics material has a nitrogen content of 10 wt.-ppm or more, 15 wt.-ppm or more, or 20 wt.-ppm or more.

9. The method according to any one of the preceding items, wherein the pre-treated liquefied waste plastics material has a nitrogen content of 2000 wt.-ppm or less, 1500 wt.-ppm or less, 1000 wt.-ppm or less, or 800 wt.-ppm or less.

10. The method according to any one of the preceding items, wherein the pre-treated liquefied waste plastics material has a sulphur content of 10 wt.-ppm or more, 15 wt.-ppm or more, or 20 wt.-ppm or more.

11. The method according to any one of the preceding items, wherein the pre-treated liquefied waste plastics material has a sulphur content of 500 wt.-ppm or less, 300 wt.-ppm or less, or 200 wt.-ppm or less.

12. The method according to any one of the preceding items, wherein the pre-treated liquefied waste plastics material has a silicon content of 10 wt.-ppm or more, 15 wt.-ppm or more, or 20 wt.-ppm or more.

13. The method according to any one of the preceding items, wherein the pre-treated liquefied waste plastics material has a silicon content of 500 wt.-ppm or less, 300 wt.-ppm or less, or 200 wt.-ppm or less.

14. The method according to any one of the preceding items, wherein the aqueous medium comprises at least 50 wt.-% water, preferably at least 70 wt.-% water, more preferably at least 85 wt.-% water or at least 90 wt.-% water, and may comprise further ingredients which are admixed with or dissolved in the water.

15. The method according to any one of the preceding items, wherein the aqueous medium having a pH of at least 7 is an alkaline aqueous medium comprising water and an alkaline substance dissolved in the water.

16. The method according to any one of the preceding items, wherein the aqueous medium having a pH of at least 7 comprises a metal hydroxide dissolved in water.

17. The method according to item 16, wherein the metal hydroxide is a hydroxide of an alkali metal and/or a hydroxide of an alkaline earth metal, preferably a hydroxide of an alkali metal.

18. The method according to any one of the preceding items, wherein the aqueous medium has a pH of 8 or more, and more preferably 9 or more.

19. The method according to any one of the preceding items, wherein the aqueous medium comprises at least 0.3 wt.-% of a metal hydroxide, more preferably at least 0.5 wt.-%, at least 1.0 wt.-%, or at least 1.5 wt.-% of a metal hydroxide.

20. The method according to any one of the preceding items, wherein the aqueous medium comprises at least 0.5 wt.-%, preferably at least 1.0 wt.-%, or at least 1.5 wt.-% of an alkali metal hydroxide.

21. The method according to any one of the preceding items, wherein contacting the liquefied waste plastics material with an aqueous medium having a pH of at least 7 in the pre-treatment step (B) is carried out at a temperature of 210° C. or more.

22. The method according to any one of the preceding items, wherein contacting the liquefied waste plastics material with an aqueous medium having a pH of at least 7 in the pre-treatment step (B) is carried out at a temperature of 220° C. or more, 240° C. or more or 260° C. or more.

23. The method according to any one of the preceding items, wherein contacting the liquefied waste plastics material with an aqueous medium having a pH of at least 7 in the pre-treatment step (B) is carried out at a temperature of 450° C. or less, preferably 400° C. or less, 350° C. or less, 320° C. or less, or 300° C. or less.

24. The method according to any one of the preceding items, wherein contacting the liquefied waste plastics material with an aqueous medium having a pH of at least 7 in the pre-treatment step (B) is carried out at a temperature in the range of 200° C. to 350° C., preferably 240° C. to 320° C., or 260° C. to 300° C.

25. The method according to any one of the preceding items, wherein the chlorine content of the liquefied waste plastics (LWP) material before pre-treatment step (B) is in the range of from 1 wt.-ppm to 4000 wt.-ppm.

26. The method according to any one of the preceding items, wherein the chlorine content of the liquefied waste plastics (LWP) material before pre-treatment step (B) is in the range of from 100 wt.-ppm to 4000 wt.-ppm.

27. The method according to any one of the preceding items, wherein the chlorine content of the liquefied waste plastics (LWP) material before pre-treatment step (B) is in the range of from 300 wt.-ppm to 4000 wt.-ppm.

28. The method according to any one of the preceding items, wherein the chlorine content of the pre-treated liquefied waste plastics (LWP) material is 400 wt.-ppm or less, preferably 300 wt.-ppm or less. steam cracker feed has an olefins content of 5.0 wt.-% or less.

29. The method according to any one of the preceding items, wherein the steam cracker feed has an olefins content of 4.0 wt.-% or less, preferably 3.5 wt.-% or less, 3.0 wt.-% or less, 2.5 wt.-% or less, 2.0 wt.-% or less, 1.0 wt.-% or less, 0.5 wt.-% or less, or 0.3 wt.-% or less.

30. The method according to any one of the preceding items, wherein the liquefied waste plastics (LWP) material provided in step (A) is a fraction of liquefied waste plastics.

31. The method according to any one of the preceding items, wherein the liquefied waste plastics (LWP) material provided in step (A) has a 5% boiling point of 25° C. or more and a 95% boiling point of 550° C. or less, preferably a 5% boiling point of 30° C. or more and a 95% boiling point of 500° C. or less, more preferably a 5% boiling point of 35° C. or more and a 95% boiling point of 400° C. or less, even more preferably a 5% boiling point of 35° C. or more and a 95% boiling point of 360° C. or less.

32. The method according to any one of the preceding items, wherein the hydrotreatment in step (C) is carried out in the presence of a hydrotreating catalyst.

33. The method according to item 32, wherein the hydrotreating catalyst in step (C) comprises at least one component selected from IUPAC group 6, 8 or 10 of the Periodic Table of Elements.

34. The method according to item 32 or 33, wherein the hydrotreating catalyst in step (C) is a supported NiMo catalyst or a supported CoMo catalyst and the support comprises alumina and/or silica, the catalyst preferably being NiMo/Al₂O₃ or CoMo/Al₂O₃.

35. The method according to any one of items 32 to 34, wherein the hydrotreating catalyst in step (C) is a supported NiMo catalyst and the support comprises alumina (NiMo/Al₂O₃).

36. The method according to any one of the preceding items, wherein the post-treatment step (D) comprise a step of blending the hydrotreated material with a paraffinic material.

37. The method according to item 36, wherein the paraffinic material has a paraffin content of 60 wt.-% or more.

38. The method according to item 36 or 37, wherein the paraffinic material has a paraffin content of 65 wt.-% or more, 70 wt.-% or more, 75 wt.-% or more, 80 wt.-% or more, 85 wt.-% or more, or 90 wt.-% or more.

39. The method according to any one of items 36 to 38, wherein the paraffinic material is at least one of a naphtha fraction, a middle distillate fraction, a VGO fraction or a LPG fraction, or a mixture of two or more thereof, preferably at least one of a naphtha fraction and a middle distillate fraction.

40. The method according to any one of items 36 to 39, wherein the paraffinic material has a paraffin content of 93 wt.-% or more, or 95 wt.-% or more.

41. The method according to any one of items 36 to 40, wherein the paraffinic material has an i-paraffin content of 5 wt.-% or more, relative to the summed amount of n-paraffins and i-paraffins in the paraffinic material taken as 100 wt.-%.

42. The method according to any one of items 36 to 41, wherein the paraffinic material has an i-paraffin content of 8 wt.-% or more, preferably 10 wt.-% or more, 15 wt.-% or more, 20 wt.-% or more, 25 wt.-% or more, 30 wt.-% or more, 35 wt.-% or more, 40 wt.-% or more, 45 wt.-% or more, 50 wt.-% or more, relative to the summed amount of n-paraffins and i-paraffins in the paraffinic material taken as 100 wt.-%.

43. The method according to any one of items 36 to 42, wherein the paraffinic material has an i-paraffin content in the range from 45 wt.-% to 70 wt.-%, preferably 50 wt.-% to 65 wt.-%, relative to the summed amount of n-paraffins and i-paraffins in the paraffinic material taken as 100 wt.-%.

44. The method according to any one of items 36 to 43, wherein the paraffinic material has a n-paraffin content in the range from 50 wt.-% to 25 wt.-%, preferably 45 wt.-% to 30 wt.-%, relative to the summed amount of n-paraffins and i-paraffins in the paraffinic material taken as 100 wt.-%.

45. The method according to any one of items 36 to 44, wherein the paraffinic material has a naphthenes content in the range from 0.01 wt.-% to 15.00 wt.-%, preferably 0.01 wt.-% to 5.00 wt.-%, relative to the total weight of the paraffinic material.

46. The method according to any one of items 36 to 45, wherein the paraffinic material has a paraffin content of 95 wt.-% or more, relative to the total weight of the paraffinic material, and an i-paraffin content in the range from 65 wt.-% to 100 wt.-%, preferably 75 wt.-% to 99 wt.-%, or 85 wt.-% to 100 wt.-%, relative to the summed amount of n-paraffins and i-paraffins in the paraffinic material taken as 100 wt.-%.

47. The method according to any one of items 36 to 46, wherein the paraffinic material is a renewable material.

48. The method according to any one of the preceding items, wherein the step (D) comprises gas-liquid separation.

49. The method according to any one of the preceding items, wherein the nitrogen content of the pre-treated liquefied waste plastics material is 2 times to 200 times (by Mole) the summed amount of the sulphur content and the chlorine content of the pre-treated liquefied waste plastics material.

50. The method according to any one of the preceding items, further comprising a step (D′) of washing the gaseous effluent from the hydrotreatment step (C) with an acidic liquid medium.

51. The method according to item 50, wherein the acidic liquid medium is a solution of an acidic substance in a solvent.

52. The method according to items 50 or 51, wherein the acidic liquid medium is a solution of an acidic substance in water.

53. The method according to item 51 or 52, wherein the acidic substance is an inorganic acidic substance.

54. The method according to item 53, wherein the inorganic acidic substance is HCl, H₂SO₂, H₂SO₃, HNO₃, H₃PO₄, H₃PO₃, or H₃PO₂ or a mixture thereof.

55. The method according to item 51 or 52, wherein the acidic substance is an organic acidic substance, preferably a carboxylic acid, such as acetic acid or formic acid.

56. The method according to any of the items 1 to 55, wherein the step (A) of providing liquefied waste plastics (LWP) material includes a step of liquefying waste plastics, preferably by thermal degradation of waste plastics, such as pyrolysis or hydrothermal liquefaction or similar process steps.

57. The method according to any of the items 1 to 56, further comprising a step (A′) of sorting waste plastics to provide sorted waste plastics, preferably removing at least 50 wt.-%, more preferably at least 55 wt.-%, at least 60 wt.-%, at least 65 wt.-%, at least 70 wt.-%, at least 75 wt.-%, at least 80 wt.-%, or at least 85 wt.-% of chlorine-containing waste plastics, such as polyvinyl chloride, PVC (relative to the original content of chlorine-containing waste plastic, such as PVC, in the waste plastics).

58. The method according to item 57, further comprising a step of liquefying the sorted waste plastics to provide liquefied sorted waste plastics (LSWP) material.

59. The method according to any of the items 1 to 58, wherein the co-feed employed in step (C) is a liquefied sorted waste plastics (LSWP) material, wherein the liquefied sorted waste plastics (LSWP) material is a material obtainable by liquefying (and optionally fractionating) sorted waste plastics.

60. The method according to item 59, wherein the amount of chlorine-containing waste plastics in the sorted waste plastics is 5 wt.-% or less, preferably 3 wt.-% or less, 2 wt.-% or less, or 1 wt.-% or less.

61. The method according to item 59 or 60, wherein the amount of PVC in the sorted waste plastics is 5 wt.-% or less, preferably 3 wt.-% or less, 2 wt.-% or less, or 1 wt.-% or less.

62. A mixture of hydrocarbons obtainable by the method according to any of the items 1 to 61.

63. Use of the mixture of hydrocarbons according to item 62 for producing chemicals and/or polymers, such as polypropylene and/or polyethylene.

### DETAILED DESCRIPTION OF THE INVENTION

The present invention relates to a method for upgrading liquefied plastics and more specifically to a two-step process for converting liquefied waste plastics into a steam cracker feed.

LWP material, such as a pyrolysis product of collected consumer plastics, contains large and varying amounts of contaminants which would be detrimental in steam cracking or in downstream processes. Such contaminants include, among others, halogens (mainly chlorine) originating from halogenated plastics (such as PVC and PTFE), sulfur originating from cross-linking agents of rubbery polymers (e.g. in end-of-life tires) and metal (e.g. Si, Al) contaminants originating from composite materials and additives (e.g. films coated with metals or metal compounds, end-of-life tires, or plastics processing aids). These contaminants may be present in elemental form, in ionic form, or as a part of organic or inorganic compounds.

These impurities/contaminants may result in coking in conventional steam cracking methods and may similarly result in (undesired) side-reactions, thus shifting the product distribution to less valuable products or even towards products which have to be disposed (i.e. waste). Similarly, these impurities may have corrosive or otherwise degrading action, thus reducing the service life of the steam cracking apparatus. In this respect, chlorine (and chlorine compounds) is one impurity which has high tendency to cause corrosion in a hydrotreatment apparatus or steam cracking apparatus. In addition, metal impurities, such as Si-containing impurities, may cause catalyst poisoning of the hydrotreater.

Moreover, the production process of LWP material usually comprises at least one kind of thermal degradation, such as pyrolysis or hydrothermal liquefaction or similar process steps. It is intrinsic to these processes that the resulting LWP has a high olefins content. The reactive extraction of pre-treatment step (B) does not significantly alter the content of olefins in LWP material (some change may occur due to removal of impurities or loss in the course of liquid-liquid separation, or the like). The hydrotreatment step (C) of the present invention reduces the content of olefins in the LWP material (and in the optional co-feed, as the case may be) and thus produces a hydrotreated material having (significantly) reduced content of olefins.

The method of the present invention comprises a step (A) of providing liquefied waste plastics (LWP) material. The mode of providing the liquefied waste plastics material is not particularly limited. That is, the liquefied waste plastics material may be produced as a part of the process of the present invention or may be purchased or procured in any other way.

The method of the present invention further comprises a pre-treatment step (B) to produce a pre-treated liquefied waste plastics (LWP) material. The step (B) comprises pre-treating the liquefied waste plastics (LWP) material by contacting the liquefied waste plastics material with an aqueous medium having a pH of at least 7 at a temperature of 200° C. or more, followed by liquid-liquid separation. In the following, pre-treatment of the LWP material by contacting the LWP material with an aqueous medium having a pH of at least 7 at a temperature of 200° C. or more, followed by liquid-liquid separation, will sometimes be simply referred to as “reactive extraction”.

By means of the reactive extraction in step (B), the amount of catalyst poisons (mainly Si) can be significantly reduced, thus increasing efficiency of hydrotreatment process as well as hydrotreatment catalyst life. Moreover, the content of other impurities is significantly reduced as well. As a consequence, hydrotreatment can be carried out efficiently and allows production of a feedstock ready to be fed to steam cracker while only relatively low amounts of hydrogen are lost in waste products (such as H₂S and HCl).

In the context of the present invention, the term “contacting” comprises physical contact and may be carried out batch-wise, e.g. using blending or mixing, or continuously, e.g. using co-current or counter-current flow, or using a combination of both. Due to easier handling, co-current flow is preferred.

The aqueous medium preferably comprises at least 50 wt.-% water, preferably at least 70 wt.-% water, more preferably at least 85 wt.-% water or at least 90 wt.-% water, and may comprise further ingredients which are admixed with or dissolved in the water.

The aqueous medium is preferably an alkaline aqueous medium comprising water and an alkaline substance (basic substance) dissolved in the water. The alkaline substance preferably is or comprises a metal hydroxide, more preferably a hydroxide of an alkali metal and/or a hydroxide of an alkaline earth metal. Preferably, the alkaline substance comprises at least an alkali metal ion, more preferably at least one of Na⁺ and K⁺. The alkaline aqueous medium preferably comprises at least 0.3 wt.-% of a metal hydroxide, more preferably at least 0.5 wt.-%, at least 1.0 wt.-% or at least 1.5 wt.-%. It is particularly preferred that the alkaline aqueous medium comprises at least 0.5 wt.-%, preferably at least 1.0 wt.-% or at least 1.5 wt.-% of an alkali metal hydroxide. According to the present invention, the aqueous medium has a pH of at least 7. Preferably, the aqueous medium has a pH of 8 or more and more preferably 9 or more.

The reactive extraction of step (B) is carried out by contacting the LWP material with the aqueous medium having a pH of at least 7 at a temperature of 200° C. or more. The temperature during reactive extraction (i.e. at the time of contacting) is preferably 210° C. or more, 220° C. or more, 240° C. or more or 260° C. or more in order to ensure sufficient reactivity and thus sufficient removal of impurities. Usually, the upper limit of the pre-treatment temperature (temperature during reactive extraction) is 600° C. so as to avoid excessive degradation. The temperature is preferably 450° C. or less, preferably 400° C. or less, 350° C. or less, 320° C. or less, or 300° C. or less. It is particularly preferred that the reactive extraction in the pre-treatment step (B) is carried out at a temperature in the range of 200° C. to 350° C., preferably 240° C. to 320° C., or 260° C. to 300° C.

The elevated temperature promotes the reactions of a reactive extraction and therefore results in faster and more efficient pre-treatment. The pre-treatment step (B) may be carried out at elevated pressure in order to ensure that the material in the pre-treatment reactor remains liquid. Useful (absolute) pressure during reactive extraction is 1 bar or more, 10 bar or more, 40 bar or more, or 60 bar or more. In order to keep equipment costs within reasonable limits the pressure should not exceed 400 bar and is preferably 200 bar or less, 150 bar or less or 100 bar or less. The pre-treatment reactor (reactive extraction reactor) may be a continuous flow reactor or a batch reactor or both and may be the same reactor as one of the reactors employed in other steps of the process, but it is preferably a different reactor or a different section of the same reactor.

In addition to the reactive extraction, other pre-treatments may be carried out in step (B), provided that step (B) does not comprise hydrotreatment. For example, the step (B) may comprise fractionation, separation or filtration in addition to the reactive extraction. The additional pre-treatments (each independently if more than one additional pre-treatment step is carried out) may be carried out either before or after the reactive extraction or may be carried out both before and after the reactive fractionation.

The pre-treatment step significantly reduces the content of contaminants in the LWP material, which makes it fit for the subsequent hydrotreatment step (C). The pre-treatment step is for reducing the amount (content) of at least one of the contaminants in the LWP material. In particular, it is preferred that the content of at least one of Si, Cl, N and S contaminants is reduced, more preferably at least the content of Cl or Si contaminants.

The method of the present invention is thus suited for a broad content range of impurities and can efficiently treat various kinds of LWP material. For example, the chlorine content of the LWP material before pre-treatment step (B) is preferably in the range of from 1 wt.-ppm to 4000 wt.-ppm, since this range can be easily handled using the method of the present invention. In order to fully develop the purification power of the present invention, the method is preferably carried out using a contaminated LWP material, and thus it is more preferably that the chlorine content of the LWP material before pre-treatment step (B) is in the range of from 100 wt.-ppm to 4000 wt.-ppm, in the range of from 200 wt.-ppm to 4000 wt.-ppm, or in the range of from 300 wt.-ppm to 4000 wt.-ppm. The pre-treatment strep (B) is adapted to reduce the content of impurities and thus the chlorine content of the pre-treated LWP material is preferably lower than the chlorine content of the LWP material before pre-treatment step (B). In particular, it is preferred that the chlorine content of the pre-treated LWP material is 400 wt.-ppm or less, more preferably 300 wt.-ppm or less, 200 wt.-ppm or less, 100 wt.-ppm or less, or 50 wt.-ppm or less.

The contaminants may be present in the LWP material in any form, e.g. in elemental form (dissolved or dispersed) or usually as organic or inorganic (usually organic) compounds.

The method of the present invention comprises a hydrotreatment step (C) to obtain a hydrotreated material. The step (C) comprises (and preferably consists of) hydrotreating the pre-treated liquefied waste plastics material, optionally in combination with (together with) a co-feed (hydrotreatment co-feed).

The (hydrotreatment) co-feed is a material which is suitable to be used as feed for hydrotreatment step (C), especially when combined with (employed together with) the pre-treated LWP material produced in step (B). The hydrotreatment co-feed may be a material which is obtainable (or obtained) from waste plastics by liquefying but may also be a renewable material (e.g. an oxygen-containing renewable material) or a mixture of those (or even a mixture of one or both of the aforementioned with another material). Preferably, the co-feed is a material obtainable (or obtained) by liquefying a sorted waste plastics material, i.e. a liquefied sorted waste plastics (LSWP) material, wherein the sorting is preferably carried out such that the amount of chlorine-containing waste plastics, such as PVC, is reduced. Preferably, the amount of chlorine-containing waste plastics in the sorted waste plastics is 5 wt.-% or less, preferably 3 wt.-% or less, 2 wt.-% or less, or 1 wt.-% or less (relative to the sorted waste plastics as a whole). In particular it is preferred that the amount of PVC in the sorted waste plastics is 5 wt.-% or less, preferably 3 wt.-% or less, 2 wt.-% or less, or 1 wt.-% or less (relative to the sorted waste plastics as a whole). In this respect, the amount (or content) of chlorine-containing waste plastics (or PVC) relates to the amount (mass) of plastic pieces (physically isolated parts) containing chlorine (or PVC).

The co-feed may be added to the pre-treated LWP material in the hydrotreatment step (C) (e.g. fed in parallel to the hydrotreatment reactor) or may be blended with the LWP material before step (C) or both.

When employing (non-pre-treated) LWP material and/or LSWP material, it is preferable that the (total) feed of step (C) comprises at least 10 wt.-% of the pre-treated LWP material of step (B), preferably at least 25 wt.-%, at least 50 wt.-%, at least 75 wt.-% or at least 90 wt.-%. The “total feed” in this context does not encompass dilution material, as mentioned below.

The step (C) may further comprise a sub-step of diluting the pre-treated material with a dilution material. The dilution sub-step allows easier temperature control during the hydrotreatment in step (C). Hence, the dilution sub-step, if present, is carried out before hydrotreatment in step (C). In order to allow appropriate temperature control, the dilution material is preferably basically inert to the hydrotreatment of step (C). In other words, it is preferably that the dilution material contain a low amount of olefins (10 wt.-% or less, preferably 5 wt.-% or less, more preferably 3 wt.-% or less and even more preferably 1 wt.-% or less, relative to the total amount of dilution material). It is particularly preferred that the dilution material comprises, essentially consists of, or consists of the hydrotreated material obtained in step (C) (or a fraction thereof). That is, the product of the hydrotreatment step (C) may be recirculated into the hydrotreatment step (C) as a dilution material.

In the present invention, it is preferably that the step (C) is the first hydrotreatment step of the process. In other words, it is preferable that the material subjected to step (C) is not a hydrotreated material and/or the process does not comprise a hydrotreating step before the step (C). In particular, it is preferably that the LWP material (in particular, the non-sorted LWP material) is not subjected to hydrotreating after having been generated (i.e. after liquefying) and before step (C).

The method of the present invention comprises a post-treatment step (D). In the step (D) the hydrotreated material is post-treated to obtain a steam cracker feed (sometimes simply referred to as “cracker feed”). Any conventional post-treatment may be applied. In particular, it is preferred that the post-treatment comprises at least one selected from separation and fractionation. In particular, the step (D) may comprise gas-liquid separation or fractionation. Preferably, at least a separation technique is carried out after hydrotreating (at least) the pre-treated LWP material, so as to remove gaseous hydrogen from the hydrotreated material.

The post-treatment step (D) may further comprise a blending sub-step of blending the hydrotreated material with an additional feed material for steam cracking. The additional feed material is preferably a paraffinic material. The additional feed material is preferably a renewable feed material or a feed material having a high content (at least 50 wt.-%) of paraffinic hydrocarbons (i.e. linear or branched alkanes), more preferably a renewable feed material having a high content (at least 50 wt.-%) of paraffinic hydrocarbons. The blending sub-step may be carried out before or after separation or fractionation or may be carried out as the only action in step (D). For example, the step (D) consists of the blending sub-step and a (gas-liquid) separation step or the blending sub-step is carried out after fractionation and/or separation. The additional feed material (cracker co-feed) may further comprise non-pre-treated LWP material (and/or non-pre-treated LSWP material, as mentioned above), preferably together with a paraffinic material as mentioned above.

The blending sub-step in step (D) may carried out in such a manner that the cracker feed meets the requirements for chlorine content and olefins content of the steam cracker, if these requirements are not yet met by the hydrotreated material. In other words, the blending sub-step preferably comprises adding the paraffinic material in such an amount that the blend (i.e. the cracker feed) meets these requirements. Preferably, the blend is intimately mixed before being fed to a steam cracker, using a batch-wise mixer or mixing means in a continuous process or both.

Preferably, the blending sub-step in step (D) comprises a first stage of determining at least one of the chlorine concentration and the olefins concentration of the hydrotreated material, a second stage of determining the amount of paraffinic material which needs to be added so as to meet the requirements for chlorine content and olefins content of the steam cracker, and a third stage of adding at least the calculated amount of the paraffinic material.

The first second and third stage may be carried out continuously or batch-wise. One or two of first second and third stage may be carried out continuously and the other one or the other two may be carried out batch-wise. It is preferred that the mode of operation (continuously or batch-wise) is the same for the first and the second stage. In case of a continuous addition, the second stage and third stage preferably provide the “amount” of the paraffinic material as a flow rate relative to the flow rate of the hydrotreated material.

The method of the invention optionally further comprises a steam cracking step (E). In step (E), the steam cracker feed is subjected to steam cracking in a steam cracker to obtain a cracker product. A co-feed (cracking co-feed) may be employed in addition to the steam cracker feed. In the present invention, steam cracking is preferred because the product distribution of the cracker product is most favourable when employing the LWP material which has been treated by steps (B), (C) and (D).

The hydrotreatment step (C) results in further purification and, in particular, is suited to reduce the content of olefins in the pre-treated LWP material (and in the hydrotreatment co-feed, as the case may be), and thus the pre-treated LWP material (or the mixture) employed in step (C) may have a broad content range of olefins. Preferably, the pre-treated liquefied waste plastics material has an olefins content of 10 wt.-% or more, 15 wt.-% or more, 20 wt.-% or more, 30 wt.-% or more, 40 wt.-% or more, or 50 wt.-% or more. Similarly, it is preferred that the pre-treated liquefied waste plastics material has an olefins content of the pre-treated liquefied waste plastics material has an olefins content of 85 wt.-% or less, 80 wt.-% or less, 70 wt.-% or less, or 65 wt.-% or less. Usually, the olefins content of the LWP material, and consequently of the pre-treated LWP material is rather high and is reduced to acceptable contents mainly by the hydrotreatment step (C).

If not mentioned otherwise, a content of a component and/or impurity is given relative to the material (e.g. the pre-treated LWP material or the steam cracker feed) as a whole being 100%.

In the present invention, the content of olefins (n-olefins, iso-olefins, diolefins, higher olefins and olefinic naphthenes), paraffins (n-paraffins and/or i-paraffins), naphthenes (excluding olefinic naphthenes) and aromatics may be determined by gas chromatography (GC) combined with a flame ionisation detector (FID) using the PIONA method (GC-FID). PIONA method is suitable for gasoline range products i.e. products boiling in the range of about 25-180° C. The content (paraffins, iso-paraffins, olefins, naphthenes, aromatics) of higher-boiling hydrocarbons, i.e. products in the range of about 180-440° C. can be determined by comprehensive gas chromatography combined with FID detector (GCxGC-FID). In case of broad boiling ranges, both methods may be used in combination.

The content of F, Cl, and Br may be determined in accordance with ASTM-D7359-18. The content of iodine (I) and sulphur (S) may be determined by XFS (X-ray fluorescence spectroscopy). Nitrogen (N) content may be determined in accordance with ASTM-D5762. Contents of phosphorous, sulphur and oxygen may be determined using known methods, e.g. P (ASTM D5185), S (ASTM D6667M) and O (ASTM D7423). The content of metal atoms may be determined using inductively coupled plasma atomic emission spectrometry (ICP-AES) based on standard ASTM D5185. The content of silicon (Si) may be determined using X-ray fluorescence (XRF) spectroscopy or using ICP-AES based on ASTM D5185. Contents of carbon (C), hydrogen (H) and others may be determined by elemental analysis using e.g. ASTM D5291.

In the present invention, the pre-treated liquefied waste plastics material (i.e. the material which is obtained in step (B)) preferably has a chlorine content of 5 wt.-ppm or more. Usually, the content of chlorine will not be reduced too much by step (B) and too much reduction would require excessive efforts. The chlorine content of the pre-treated liquefied waste plastics material is preferably 10 wt.-ppm or more, 15 wt.-ppm or more, or 20 wt.-ppm or more. Furthermore, the chlorine content of the pre-treated liquefied waste plastics material is preferably 1000 wt.-ppm or less, 600 wt.-ppm or less, 400 wt.-ppm or less, 300 wt.-ppm or less, 200 wt.-ppm or less, or 100 wt.-ppm or less, since too high a chlorine content may be detrimental. A minimum content of sulphur may be helpful in the hydrotreatment step in order to ensure full catalyst efficiency. Hence, it is further preferred that the pre-treated liquefied waste plastics material has a sulphur content of 10 wt.-ppm or more, 15 t.-ppm or more, or 20 wt.-ppm or more. If necessary, spiking may be carried out after the step (B) so as to ensure a minimum content of sulphur in the step (C) (i.e. a sulphur-containing material may be used as a further co-feed in the step (C)). It is similarly preferred that the pre-treated liquefied waste plastics material has a sulphur content of 500 wt.-ppm or less, 300 wt.-ppm or less, or 200 wt.-ppm or less.

Silicon-containing impurities may cause catalyst poising hydrotreating catalyst. Thus, the present invention employs reactive extraction of step (B) for reducing the content of Si-based impurities. In particular, it is preferred that the pre-treated liquefied waste plastics material has a silicon content of 500 wt.-ppm or less, 300 wt.-ppm or less, or 200 wt.-ppm or less. Such limits can be easily achieved using the reactive extraction of the present invention and thus no complicated purification procedures are required.

In order to keep the reactive extraction process simple, it is acceptable that the pre-treated liquefied waste plastics material has a silicon content of 3 wt.-ppm or more, 5 wt.-ppm or more, 10 wt.-ppm or more, 15 wt.-ppm or more, or 20 wt.-ppm or more. Contents of up to about 10 wt.-ppm may be tolerated by the hydrotreatment catalyst. If the content exceeds 10 wt.-ppm, it is preferable to employ a dilution material (as described above) and/or to employ guard bed(s) before (upstream) the hydrotreatment catalyst.

In the present invention, the steam cracker feed (cracker feed) preferably meets the requirements for chlorine content and olefins content of a steam cracker.

The cracker feed that meets the requirements for chlorine content and olefins content of a steam cracker preferably has a chlorine content of 10 ppm by weight (wt.-ppm) or less. Chlorine impurities are very harmful for the steam cracker equipment and thus should be rigorously controlled. More preferably, the cracker feed that meets the requirements for chlorine content and olefins content of the steam cracker has a chlorine content of 8 wt.-ppm or less, 6 wt.-ppm or less, 5 wt.-ppm or less, 4 wt.-ppm or less, or 3 wt.-ppm or less.

The cracker feed that meets the requirements for chlorine content and olefins content of the steam cracker preferably has an olefins content of 5.0 wt.-% or less. Olefins tend to cause coking or fouling in the steam cracker and thus their content should be controlled to a relatively low level. Thus, more preferably, the cracker feed that meets the requirements for chlorine content and olefins content of the steam cracker has an olefins content of 4.0 wt.-% or less, 3.5 wt.-% or less, 3.0 wt.-% or less, 2.5 wt.-% or less, 2.0 wt.-% or less, 1.0 wt.-% or less, 0.5 wt.-% or less, or 0.3 wt.-% or less. Consequently, it is preferred that the step (C) be adjusted such that the above-mentioned olefin-content is achieved already for the hydrotreated material. The hydrotreatment step (C) can be easily adjusted to remove virtually all of the olefins (i.e. full hydrogenation of olefinic bonds).

If not mentioned otherwise, a content of a feed component and/or impurity is given relative to the feed as a whole being 100%.

In the present invention, the pre-treated liquefied waste plastics material preferably has a nitrogen content of 10 wt.-ppm or more, 15 wt.-ppm or more, or 20 wt.-ppm or more. The nitrogen content may be 100 wt.-ppm or more, 150 wt.-ppm or more or 200 wt.-ppm or more. It is further preferred that the pre-treated liquefied waste plastics material has a nitrogen content of 2000 wt.-ppm or less, 1500 wt.-ppm or less, 1000 wt.-ppm or less, or 800 wt.-ppm or less.

Using the pre-treatment step (B) of the present invention, nitrogen removal is not always as effective as the chlorine removal, although nitrogen removal rates of 50 to 90% (by weight) can usually be achieved. Since chlorine and nitrogen are often the main contaminants in LWP material, especially in LWP derived from post-consumer plastics, the considerably high removal rate of chlorine contaminants results in an excess of nitrogen contaminants. That is, the content of nitrogen (by Mole) in the pre-treated LWP material may often be at least 2 times and up to 100 times the summed amount (by Mole) of sulphur and chlorine. Accordingly, other than usual in the hydrotreatment of LWP material, the gaseous effluent of the hydrotreatment apparatus is basic rather than acidic. That is, while the pre-treatment effectively reduces the amount of chlorine in the LWP material (which is converted to HCl in the hydrotreatment and usually causes the gaseous effluent to be strongly acidic), the content of nitrogen is often not reduced that much.

Thus, as a result of the pre-treatment step (B) of the present invention and the subsequent hydrotreatment, a surprisingly basic gaseous effluent may be produced in the hydrotreatment step (C). The present invention thus preferably further comprises a step (D′) of washing the gaseous effluent from the hydrotreatment step (C) with an acidic liquid medium.

In order to provide the gaseous effluent, in particular in a continuous flow hydrotreatment, the step (D) preferably comprises gas-liquid separation. The acidic liquid medium is preferably a solution of an acidic substance in a solvent.

The solvent may be water. The acidic substance may an inorganic acidic substance or an organic acidic substance. The inorganic acidic substance may be HCl, H₂SO₂, H₂SO₃, HNO₃, H₃PO₄, H₃PO₃, or H₃PO₂ or a mixture thereof. The organic acidic substance may be a carboxylic acid, such as acetic acid or formic acid.

In the present invention, the pre-treatment step (B) comprises reactive extraction in which the LWP material is chemically modified in the course of the extraction step. The present inventors found that by contacting a contaminated material with an aqueous medium having a pH of 7 or more, the aqueous medium can act as a reactive extraction medium, thus converting contaminants (including organic compounds) into water-soluble contaminants (and other products which may be water-soluble or water-insoluble) and these can thus be extracted together with the water.

In particular, the pre-treatment step (B) comprises contacting (e.g. blending) the LWP material with an aqueous medium having a pH of at least 7 at a temperature of 200° C. or more, followed by liquid-liquid separation. Phase separation (in the course of liquid-liquid separation) may be induced phase separation, e.g. using physical methods (such as centrifugation) or chemical methods (such as addition of separation aids, e.g. solvent(s) other than aqueous medium, additional amounts of the aqueous medium or an aqueous medium having a different concentration of additional ingredients, such as alkaline substance), or non-induced phase separation, such as gravity-driven phase separation.

Preferably, the mass ratio between the amount (Aq) of aqueous medium employed in the pre-treatment step (B) and the amount (LW) of LWP material fed to the pre-treatment step (B), Aq:LW, is in the range of 1:10 to 9:1. This means that the content of Ex relative to Aq+LW is about 9.09 to 90 wt.-%. Hence, good impurity removal efficiency can be achieved. The ratio is preferably 1:5 to 5:1, more preferably 1:5 to 2:1, or 1:5 to 1.5:1.

In the present invention, it is preferred that no hydrogen is added in the pre-treatment step (B) and/or no hydrotreating catalyst is present. That is, the pre-treatment step does not comprise or a hydrotreatment process in which the impurities are removed by hydrotreatment e.g. as HCl in the case of chlorine, or resulting in saturation of olefins. Preferably, at least one of hydrogen and hydrotreating catalyst is absent in the pre-treatment step (at least at the same time), more preferably both are absent.

More preferably, no hydrogen gas (including dissolved hydrogen gas) is present during the pre-treatment step (B).

In the present invention, it is preferable that the ratio between the bromine number (BN2) of the pre-treated liquefied waste plastics (LWP) material and the bromine number (BN1) of the liquefied waste plastics (LWP) material, BN2/BN1 is 0.90 or more, preferably 0.95 or more. In the present invention, the bromine number can be determined in accordance with ASTM D1159-07 (2017).

The bromine number (BN2) of the pre-treated liquefied waste plastics (LWP) material refers to the bromine number immediately after the pre-treatment step (B). The bromine number (BN1) of the liquefied waste plastics (LWP) material refers to the bromine number immediately before the pre-treatment step (B)). In other words, in this embodiment, the pre-treatment does not significantly reduce the amount of olefins. This similarly means that the pre-treatment step substantially does not result in saturation of olefins. That is, although olefins can be harmful for the steam cracker, the present invention employs the hydrotreatment step (C) and thus can meet the olefins restrictions of the steam cracker. Usually the olefins content will not increase by the pre-treatment (except for minor effects due to removal of impurity components) and thus the upper limit of the ratio is may be 1.2 and is preferably 1.1 or 1.0.

The liquefied waste plastics (LWP) material provided in step (A) may be a fraction of liquefied waste plastics. The step (A) may comprise a sub-step (A2) of fractionating liquefied waste plastics, but the fraction of liquefied waste plastics may similarly be purchased or provided by other means.

The step (A) may further comprise a sub-step (A1) of liquefying waste plastics, either alone or together with sub-step (A2). The liquefying may be carried out by any known method such as pyrolysis, including fast pyrolysis and hydropyrolysis.

The liquefied waste plastics (LWP) material provided in step (A) preferably has a 5% boiling point of 25° C. or more and a 95% boiling point of 550° C. or less. The 5% and 95% boiling points of the LWP material may be determined in accordance with ASTM D2887-16.

The hydrotreatment step (C) comprises hydrotreating the pre-treated liquefied waste plastics material (optionally together with a co-feed) to obtain a hydrotreated material. Preferably, the hydrotreatment in step (C) is carried out in the presence of a hydrotreating catalyst. Of course, the hydrotreatment is carried out in the presence of hydrogen (hydrogen gas, H₂).

The hydrotreating catalyst in step (C) preferably comprises at least one component selected from IUPAC group 6, 8 or 10 of the Periodic Table of Elements. For example, the hydrotreating catalyst in step (C) is a supported NiMo catalyst or a supported CoMo catalyst and the support is alumina and/or silica. The catalyst is preferably NiMo/Al₂O₃ or Co—Mo/Al₂O₃. More preferably, the hydrotreating catalyst in step (C) is a supported NiMo catalyst and the support is alumina (NiMo/Al₂O₃).

As said above, the step (C) may comprise a sub-step of diluting the pre-treated material with a dilution material. The dilution material may be any material which is not (substantially) modified by the conditions of step (C). Preferably, the dilution material is or comprises the effluent of hydrotreatment step (C) or a fraction thereof. Furthermore, the step (D) may comprise a sub-step of blending the hydrotreated material with an additional feed material for steam cracking. The additional feed material may be a paraffinic material. The paraffinic material is preferably a material having a high content (at least 50 wt.-%) of paraffinic hydrocarbons

Preferably, the paraffinic material contains at least 90 wt.-% of compounds having 5 or more carbon atoms (C5-plus material). In other words, it is preferred that at least 90 wt.-% of the paraffinic material is made up of compounds having 5 or more carbon atoms. In particular, the paraffinic material should not have too much C4-minus material (compounds having 4 or less carbon atoms), since these components are volatile and thus handling is difficult, especially when blending the material with the hydrotreated material. Moreover, C5-plus material has more pronounced effects on the product distribution of a steam cracking step. It is particularly preferable that the paraffinic material contains at least 90 wt.-% of compounds having a carbon number (number of carbon atoms) in the range of from 5 to 40.

The paraffinic material preferably has a 5% boiling point (based on ASTM D86) in the range of from 20° C. to 300° C. In other words, the paraffinic material may have a boiling start point (5% boiling point) which is comparable to that of usual (e.g. fossil) fuel fractions.

The paraffinic material is preferably at least one of a naphtha fraction, a middle distillate fraction, a VGO fraction or a LPG fraction, or a mixture of two or more thereof, preferably at least one of a naphtha fraction and a middle distillate fraction.

Preferably only one of these fractions is employed as a paraffinic material. In the context of the present invention, a naphtha fraction preferably has a boiling start point of 25° C. or more and a boiling end point of 200° C. or less (ASTM D86); a middle distillate fraction preferably has a boiling start point of 180° C. or more and a boiling end point of 360° C. or less (ASTM D86); a VGO fraction preferably has a boiling start point of 360° C. or more (ASTM D2887-16); and a LPG fraction preferably has a boiling end point of 25° C. or less (ASTM D86).

In the present invention, it is preferred that the paraffinic material has a paraffin content of 60 wt.-% or more, since the beneficial effects on the steam cracking product distribution will thus be more pronounced. In the context of the present invention, the paraffins content refers to the sum of contents of n-paraffins and i-paraffins and is determined relative to the paraffinic material as a whole. More preferably, the paraffinic material has a paraffin content of 65 wt.-% or more, 70 wt.-% or more, 75 wt.-% or more, 80 wt.-% or more, 85 wt.-% or more, 90 wt.-% or more, 93 wt.-% or more, or 95 wt.-% or more.

Even better results can be achieved when employing a paraffinic material containing i-paraffins (iso-paraffins). It is preferred that the paraffinic material has an i-paraffin content of 5 wt.-% or more. In the present invention, the i-paraffin content is determined relative to total paraffins content of the paraffinic material taken as 100 wt.-%. More preferably, the paraffinic material has an i-paraffin content of 8 wt.-% or more, 10 wt.-% or more, preferably 15 wt.-% or more, 20 wt.-% or more, 25 wt.-% or more, 30 wt.-% or more, 35 wt.-% or more, 40 wt.-% or more, 45 wt.-% or more, 50 wt.-% or more.

In an embodiment, the paraffinic material has an i-paraffin content in the range from 45 wt.-% to 70 wt.-%, preferably 50 wt.-% to 65 wt.-%.

In another embodiment, the paraffinic material has a paraffin content of 95 wt.-% or more and an i-paraffin content in the range from 65 wt.-% to 100 wt.-%, preferably 75 wt.-% to 99 wt.-%, 80 wt.-% to 99 wt.-%, or 85 wt.-% to 99 wt.-%.

In a further embodiment, the paraffinic material has a paraffin content of 95 wt.-% or more and an i-paraffin content in the range from preferably 80 wt.-% to 100 wt.-%, or 85 wt.-% to 99 wt.-%.

The paraffinic material preferably has a n-paraffin content in the range from 50 wt.-% to 25 wt.-%, preferably 45 wt.-% to 30 wt.-%. The paraffinic material preferably has a naphthenes content in the range from 0 wt.-% to 15.00 wt.-%, preferably 0.01 wt.-% to 5.00 wt.-%.

In view of sustainability, it is particularly preferable that the paraffinic material be a renewable material.

Renewable in the context of the present invention means a renewable content (content of bio-material; more specifically carbon derived from bio-material, i.e. bio-carbon) of 95 wt.-% or more. The content of bio-carbon (bio-material) may be determined in accordance with ASTM D 6866-18.

In particular, a renewable material obtained by hydrotreating (hydrodeoxygenation) of triglycerides and/or fatty acids and optionally isomerisation, followed by fractionation so as to obtain a renewable material fraction is preferred in the present invention. Such a material can provide a well-defined and quite uniform carbon number distribution which has been found to further improve the product distribution of the method of the present invention.

Especially renewable diesel (diesel fraction obtained by hydrotreating triglycerides, followed by isomerisation and fractionation) is a highly potential blending component for LWP for several reasons. First, LWP and renewable diesel are complementary because both feedstocks can fulfil several sustainability targets. Second, renewable diesel is an excellent blending feedstock having very low impurity, olefin or aromatic levels. Therefore, renewable diesel can be used to reduce the impurity levels and boost the performance of LWP and thus make it more suitable feedstock, especially for naphtha crackers.

The steam cracking process of the present invention may be carried out under usual conditions known in the art. Since the core of the present invention is the steam cracker feed material, the steam cracking process as such this process is not described in full detail and the reader may refer to the prior art for suitable variations.

In general, the steam cracking step is performed at elevated temperatures, preferably in the range of from 650 to 1000° C., more preferably of from 750 to 850° C. Steam is mixed with the hydrocarbon feed before the cracking zone, making the cracking reaction more robust against impurities and coke precursors. The cracking usually occurs in the absence of oxygen. The residence time at the cracking conditions is very short, typically on the order of milliseconds. From the cracker, a cracker effluent is obtained that may comprise aromatics, olefins, hydrogen, water, carbon dioxide and other hydrocarbon compounds. The specific products obtained depend on the composition of the feed, the hydrocarbon-to-steam ratio, and the cracking temperature and furnace residence time. The cracked products from the steam cracker are then usually passed through one or more heat exchangers, often referred to as TLE's, to rapidly reduce the temperature of the cracked products. The TLE's preferably cool the cracked products to a temperature in the range of from 400 to 550° C.

The (optional) step (E) provides a cracker product (also referred to as “cracked product” or “cracking product”). In the present invention, the term “cracking products” (or “cracked products” or “cracker products”) may refer to products obtained directly after the steam cracking step (also referred to as “thermal cracking step” in the following), or to derivatives thereof, i.e. “cracking products” as used herein refers to the hydrocarbon species in the mixture of hydrocarbons, and their derivatives. “Obtained directly after the steam cracking step” may be interpreted as including optional separation and/or purification steps. As used herein, the term “cracking product” may also refer to the mixture of hydrocarbons obtained directly after the steam cracking step as such.

The present invention provides a mixture of hydrocarbons obtainable by the method according to the invention. The mixture of hydrocarbons corresponds to the mixture which is directly obtained after thermal cracking without further purification.

The present invention further provides use of the mixture of hydrocarbons for producing chemicals and/or polymers. Use of the mixture of hydrocarbons for producing chemicals and/or polymers may comprise a separation step to separate at least one hydrocarbon compound from the mixture of hydrocarbons.

The cracking products described herein are examples of cracking products obtainable with the present invention. The cracking products of a certain embodiment may include one or more of the cracking products described in the following.

In a preferred embodiment, the cracking products include one or more of hydrogen, methane, ethane, ethene, propane, propene, propadiene, butane and butylenes, such as butene, iso-butene, and butadiene, C5+ hydrocarbons, such as aromatics, benzene, toluene, xylenes, and C5-C18 paraffins or olefins, and their derivatives.

Such derivatives are, for example, methane derivatives, ethene derivatives, propene derivatives, benzene derivatives, toluene derivatives, and xylene derivatives, and their derivatives.

Methane derivatives include, for example, ammonia, methanol, phosgene, hydrogen, oxochemicals and their derivatives, such as methanol derivatives. Methanol derivatives include, for example, methyl methacrylate, polymethyl methacrylate, formaldehyde, phenolic resins, polyurethanes, methyl-tert-butyl ether, and their derivatives.

Ethene derivatives include, for example, ethylene oxide, ethylene dichloride, acetaldehyde, ethylbenzene, alpha-olefins, and polyethylene, and their derivatives, such as ethylene oxide derivatives, ethylbenzene derivatives, and acetaldehyde derivatives. Ethylene oxide derivatives include, for example, ethylene glycols, ethylene glycol ethers, ethylene glycol ethers acetates, polyesters, ethanol amines, ethyl carbonates and their derivatives. Ethylbenzene derivatives include, for example, styrene, acrylonitrile butadiene styrene, styrene-acrylonitrile resin, polystyrene, unsaturated polyesters, and styrene-butadiene rubber, and their derivatives. Acetaldehyde derivatives include, for example, acetic acid, vinyl acetate monomer, polyvinyl acetate polymers, and their derivatives. Ethyl alcohol derivatives include, for example, ethyl amines, ethyl acetate, ethyl acrylate, acrylate elastomers, synthetic rubber, and their derivatives. Further, ethene derivatives include polymers, such as polyvinyl chloride, polyvinyl alcohol, polyester such as polyethylene terephthalate, polyvinyl chloride, polystyrene, and their derivatives.

Propene derivatives include, for example, isopropanol, acrylonitrile, polypropylene, propylene oxide, acrylic acid, allyl chloride, oxoalcohols, cumens, acetone, acrolein, hydroquinone, isopropylphenols, 4-hethylpentene-1, alkylates, butyraldehyde, ethylene-propylene elastomers, and their derivatives. Propylene oxide derivatives include, for example, propylene carbonates, allyl alcohols, isopropanolamines, propylene glycols, glycol ethers, polyether polyols, polyoxypropyleneamines, 1,4-butanediol, and their derivatives. Allyl chloride derivatives include, for example, epichlorohydrin and epoxy resins. Isopropanol derivatives include, for example, acetone, isopropyl acetate, isophorone, methyl methacrylate, polymethyl methacrylate, and their derivatives. Butyraldehyde derivatives include, for example, acrylic acid, acrylic acid esters, isobutanol, isobutylacetate, n-butanol, n-butylacetate, ethylhexanol, and their derivatives. Acrylic acid derivatives include, for example, acrylate esters, polyacrylates and water absorbing polymers, such as super absorbents, and their derivatives.

Butylene derivatives include, for example, alkylates, methyl tert-butyl ether, ethyl tert-butyl ether, polyethylene copolymer, polybutenes, valeraldehyde, 1,2-butylene oxide, propylene, octenes, sec-butyl alcohol, butylene rubber, methyl methacrylate, isobutylenes, polyisobutylenes, substituted phenols, such as p-tert-butylphenol, di-tert-butyl-p-cresol and 2,6-di-tert-butylphenol, polyols, and their derivatives. Other butadiene derivatives may be styrene butylene rubber, polybutadiene, nitrile, polychloroprene, adiponitrile, acrylonitrile butadiene styrene, styrene-butadiene copolymer latexes, styrene block copolymers, styrene-butadiene rubber.

Benzene derivatives include, for example, ethyl benzene, styrene, cumene, phenol, cyclohexane, nitrobenzene, alkylbenzene, maleic anhydride, chlorobenzene, benzene sulphonic acid, biphenyl, hydroquinone, resorcinol, polystyrene, styrene-acrylonitrile resin, styrene-butadiene rubber, acrylonitrile-butadiene-styrene resin, styrene block copolymers, bisphenol A, polycarbonate, methyl diphenyl diisocyanate and their derivatives. Cyclohexane derivatives include, for example, adipic acid, caprolactam and their derivatives. Nitrobenzene derivatives include, for example, aniline, methylene diphenyl diisocyanate, polyisocyanates and polyurethanes. Alkylbenzene derivatives include, for example, linear alkybenzene. Chlorobenzene derivatives include, for example, polysulfone, polyphenylene sulfide, and nitrobenzene. Phenol derivatives include, for example, bisphenol A, phenol form aldehyde resins, cyclohexanone-cyclohexenol mixture (KA-oil), caprolactam, polyamides, alkylphenols, such as p-nonoylphenol and p-dedocylphenol, ortho-xylenol, aryl phosphates, o-cresol, and cyclohexanol.

Toluene derivatives include, for example, benzene, xylenes, toluene diisocyanate, benzoic acid, and their derivatives.

Xylene derivatives include, for example, aromatic diacids and anhydrates, such as terephthalic acid, isophthalic acid, and phthalic anhydrate, and phthalic acid, and their derivatives. Derivatives of terephthalic acid include, for example, terephthalic acid esters, such as dimethyl terephthalate, and polyesters, such as polyethylene terephthalate, polytrimethylene terephthalate, polybutylene terephthalate and polyester polyols. Phthalic acid derivatives include, for example, unsaturated polyesters, and PVC plasticizers. Isophthalic acid derivatives include, for example, unsaturated polyesters, polyethylene terephthalate co-polymers, and polyester polyols.

The hydrocarbons obtained or obtainable with the method according to the present invention are particularly suitable as raw materials for conventional petrochemistry, and polymer industry. Specifically, the mixture of hydrocarbons obtained from the present invention show a product distribution which is similar to, and even favourable over, the product distribution obtained from thermal (steam) cracking of conventional raw material, i.e. neat fossil raw material. Thus, these hydrocarbons can be added to the known value-added chain while no significant modifications of production processes are required.

The cracking products of the current invention may be used in a wide variety of applications. Such applications are, for example, consumer electronics, composites, automotive, packaging, medical equipment, agrochemicals, coolants, footwear, paper, coatings, adhesives, inks, pharmaceuticals, electric and electronic appliances, sport equipment, disposables, paints, textiles, super absorbents, building and construction, fuels, detergents, furniture, sportwear, solvents, plasticizers and surfactants.

### EXAMPLES

Steam cracking was carried out under varying temperature conditions using LWP fractions, and fossil naphtha.

### Example 1

A middle distillate fraction of a LWP material (“LWP-MD”; 5-95 wt.-% distillation range 172-342° C.) was pre-treated and subsequently subjected to hydrotreatment.

Pre-treatment was carried out in a stirred batch reactor using 300 parts LWP MD and 200 parts 2 wt. % aqueous NaOH. The reactor was sealed at ambient pressure and temperature and then heated up to 240° C., holding this temperature for 30 minutes and then allowing the reactor to cool down again. The water phase was roughly decanted from the organic phase, followed by centrifugation (20° C., 4300 rpm, 30 minutes) of the organic phase and recovering the separated organic phase.

Thus pre-treated LWP material was then subjected to hydrotreatment. The hydrotreatment was carried out in a 450 ml autoclave reactor at 300° C. and 90 bar H₂ pressure using a sulfided NiMo catalyst (supported on alumina). The reaction time was 6 h. A constant H₂ flow of 40 I/h was maintained during the reaction. The amount of the pre-treated LWP material was 215 g and the catalyst amount was 43 g. The catalyst was placed directly into the liquid, and gas-liquid separation as well as catalyst removal via filtration were carried out (post-treatment). The results are presented in Table 1 and clearly shows that the heteroatoms (impurities) which remained in the LWP middle distillate fraction after the pretreatment step were effectively removed in the hydrotreatment step.

TABLE 1

| | | Pre-treated | Decrease | Hydrotreated | |
|---|---|---|---|---|---|
| | LWP-MD | LWP material | (%) | material | |
| N (mg/kg) | 810 | 350 | 57 | 0.6 | mg/l^{a} |
| Cl (mg/kg) | 590 | 179 | 70 | <8 | |
| Br (mg/kg) | 325 | 96 | 70 | <5 | |
| S (mg/kg) | 695 | 560 | 19 | <5 | |
| Si (mg/kg) | 250 | 65 | 69 | <9 | |
| Br number | — | 57 g/100 g | — | 68 | mg/100 g |

^{a}N content of hydrotreated product analysed using ASTMD4629

The decrease in the amount of unsaturated compounds in the LWP middle distillate fraction was observable through bromine (Br) number/index analyses according to ISO3839 (for pre-treated LWP material; g/100 g) and ASTMD2710 (for hydrotreated material; mg/100 g). The essentially complete saturation of olefinic material could further be confirmed by infrared (IR) spectroscopy, according to which the hydrotreated LWP no longer contained noticeable amounts olefinic hydrocarbons.

The thus obtained cracker feed was suited as a drop-in steam cracker feed for steam cracking together with a fossil or renewable cracker feed.

### Example 2

The procedure of Example 1 was repeated in a similar manner but using crude (undistilled) LWP material as an initial feed.

The obtained hydrotreated material was subjected to fractionation after filtering off the catalyst to obtain several fractions, of which at least the middle distillate fraction contained low amounts of impurities (results not shown), similar to the steam cracker feed of Example 1.

### Example 3

A gasoline fraction of a LWP material (LWP-gasoline; 5%-95% boiling range of 85-174° C.) was pre-treated using the same procedure as in Example 1. The pre-treatment significantly reduced the amount of impurities while the olefins content slightly increased, as shown in Table 2.

The resulting pre-treated LWP material was subjected to hydrotreatment under the same conditions as in Example 1. The product was analyzed by IR spectroscopy, confirming the absence of olefins.

TABLE 2

| | | Pre-treated | Decrease | Hydrotreated |
|---|---|---|---|---|
| | LWP-gasoline | LWP material | (%) | material |
| N (mg/kg) | 500 | 25 | 95 | — |
| Cl (mg/kg) | 590 | 36 | 94 | — |
| Br (mg/kg) | 210 | 7 | 97 | — |
| S (mg/kg) | 89 | 80 | 10 | — |
| Olefins | 61 wt.-% | 66 wt.% | — | ~0 wt.-% |

### Example 4

The hydrotreated material of Example 1 was subject to steam cracking at coil outlet temperatures (COT) of 820° C., 850° C. and 880° C. Water to hydrocarbon ratio (gH2O/HC) was 0.5. The water to hydrocarbon ration was adjusted by feeding the water fraction at the rate of 0.075 kg/h and the hydrocarbon fraction at the rate of 0.15 kg/h. The coil outlet pressure (COP) was 1.7 bar(a) in all experiments. Sulphur content (related to the HC content) was adjusted by dimethyl disulfide (DMDS) to 250 ppm. For reference, fossil naphtha was steam cracked under the same conditions.

The yields of ethylene, propylene and CO are shown in Table 3.

TABLE 3

| | Hydrotreated | | | Fossil | | |
|---|---|---|---|---|---|---|
| Sample | material | | | naphtha | | |
| COT [° C.] | 820 | 850 | 880 | 820 | 850 | 880 |
| COP [bar(a)] | 1.7 | 1.7 | 1.7 | 1.7 | 1.7 | 1.7 |
| Water ratio | 0.5 | 0.5 | 0.5 | 0.5 | 0.5 | 0.5 |
| kgH2O/kgHC | | | | | | |
| Yields [wt. %] | | | | | | |
| H2 | 0.61 | 0.68 | 0.81 | 0.76 | 1.00 | 1.19 |
| ΣC4− | 73.1 | 71.7 | 71.28 | 63.37 | 77.84 | 74.7 |
| ΣC5+ | 26.9 | 28.3 | 28.72 | 36.63 | 22.16 | 25.3 |
| C1-C4 species | | | | | | |
| CH4 | 9.93 | 11.4 | 13.3 | 9.85 | 14.01 | 15.72 |
| CO | 0.17 | 0.13 | 0.15 | 0.00 | 0.04 | 0.07 |
| C2H4 | 31.13 | 31.99 | 33.89 | 21.31 | 29.03 | 30.85 |
| C3H6 | 15.67 | 13.97 | 11.7 | 16.03 | 17.51 | 13.96 |
| 1-C4H8 | 3.24 | 1.62 | 0.56 | 2.67 | 2.07 | 0.99 |
| 1,3-C4H6 | 5.5 | 5.39 | 4.77 | 4.37 | 4.95 | 4.52 |
| BTX | | | | | | |
| Benzene | 4.59 | 4.94 | 5.63 | 4.47 | 5.78 | 9.95 |
| Toluene | 2.42 | 2.61 | 2.64 | 0.12 | 0.12 | 0.54 |
| Xylenes | 0.63 | 0.65 | 0.62 | 0.64 | 1.14 | 1.95 |

The results show that the hydrotreated material has good properties as a drop-in steam cracker feed which are comparable to those of fossil naphtha, though being obtained from highly contaminated waste products and thus contributing to sustainability of the resulting products.

### Example 5

The pre-treatment procedure of Example 1 was repeated using a crude undistilled LWP sample (LWP-crude; 5-95 wt.-% distillation range 67-476° C.). As a result, it was found that the effect of the pre-treatment procedure is particularly high for removing chlorine impurities while the removal efficiency of nitrogen and sulfur is more limited. For this sample, the initial sulfur content was moderate while the initial nitrogen content was rather high. Thus, the resulting pre-treated material had much higher nitrogen content (both on a mass basis and on a molar basis) than the summed amount of sulfur and chlorine content. The results are shown in Table 4.

TABLE 4

| | | Pre-treated |
|---|---|---|
| | LWP-crude | LWP-crude |
| N (mg/kg) | 850 | 470 |
| Cl (mg/kg) | 75 | 8 |
| Br (mg/kg) | 15 | <5 |
| S (mg/kg) | 321 | 240 |
| Si (mg/kg) | 12 | <8 |

Accordingly, other than usual in the hydrotreatment of LWP material, the gaseous effluent of the hydrotreatment step surprisingly had basic character and the gaseous effluent was post-treated using an acidic liquid medium (aqueous solution of sulfuric acid)

1-15. (canceled)

16. A method for upgrading liquefied waste plastics, the method comprising:
a step (A) of providing liquefied waste plastics (LWP) material,
a step (B) comprising pre-treating the liquefied waste plastics material by contacting the liquefied waste plastics material with an aqueous medium having a pH of at least 7 at a temperature of 200° C. or more, followed by liquid-liquid separation, to produce a pre-treated liquefied waste plastics material,
a step (C) comprising hydrotreating the pre-treated liquefied waste plastics material, optionally in combination with a co-feed, to obtain a hydrotreated material, and
a step (D) of post-treating the hydrotreated material to obtain a steam cracker feed.

17. The method according to claim 16, further comprising a step (E) of subjecting the steam cracker feed to steam cracking.

18. The method according to claim 16, wherein the pre-treated liquefied waste plastics material has a chlorine content of 5 wt.-ppm or more, 10 wt.-ppm or more, 15 wt.-ppm or more, or 20 wt.-ppm or more, and/or wherein the pre-treated liquefied waste plastics material has an olefins content of 10 wt.-% or more, 15 wt.-% or more, 20 wt.-% or more, 30 wt.-% or more, 40 wt.-% or more, or 50 wt.-% or more.

19. The method according to claim 16, wherein the pre-treated liquefied waste plastics material has a silicon content of 500 wt.-ppm or less, 300 wt.-ppm or less, or 200 wt.-ppm or less.

20. The method according to claim 16, wherein the aqueous medium having a pH of at least 7 is an alkaline aqueous medium comprising water and an alkaline substance dissolved in the water, and preferably comprises a metal hydroxide dissolved in water, wherein the metal hydroxide is preferably a hydroxide of an alkali metal and/or a hydroxide of an alkaline earth metal, more preferably a hydroxide of an alkali metal.

21. The method according to claim 16, wherein the aqueous medium comprises at least 0.3 wt.-% of a metal hydroxide, preferably at least 0.5 wt.-%, at least 1.0 wt.-%, or at least 1.5 wt.-% of a metal hydroxide.

22. The method according to claim 16, wherein contacting the liquefied waste plastics material with an aqueous medium having a pH of at least 7 in the pre-treatment step (B) is carried out at a temperature of 210° C. or more, preferably 220° C. or more, 240° C. or more or 260° C. or more.

23. The method according to claim 16, wherein the steam cracker feed has an olefins content of 5.0 wt.-% or less, preferably 4.0 wt.-% or less, 3.5 wt.-% or less, 3.0 wt.-% or less, 2.5 wt.-% or less, 2.0 wt.-% or less, 1.0 wt.-% or less, 0.5 wt.-% or less, or 0.3 wt.-% or less.

24. The method according to claim 16, wherein the liquefied waste plastics (LWP) material is obtainable by a process comprising at least one kind of thermal degradation.

25. The method according to claim 16, wherein the liquefied waste plastics (LWP) material provided in step (A) is a fraction of liquefied waste plastics.

26. The method according to claim 16, wherein the hydrotreatment in step (C) is carried out in the presence of a hydrotreating catalyst, wherein the hydrotreating catalyst in step (C) is preferably a supported NiMo catalyst or a supported CoMo catalyst and the support comprises alumina and/or silica, the catalyst more preferably being NiMo/Al₂O₃ or CoMo/Al₂O₃.

27. The method according to claim 16, wherein the post-treatment step (D) comprise a step of blending the hydrotreated material with a paraffinic material, wherein the paraffinic material preferably has a paraffin content of 60 wt.-% or more, more preferably 65 wt.-% or more, 70 wt.-% or more, 75 wt.-% or more, 80 wt.-% or more, 85 wt.-% or more, or 90 wt.-% or more, and wherein the paraffinic material is preferably a renewable material.

28. The method according to claim 16, wherein the step (D) comprises at least one selected from fractionation and separation, such as gas-liquid separation.

29. The method according to claim 16, further comprising a step (D′) of washing the gaseous effluent from the hydrotreatment step (C) with an acidic liquid medium.

30. The method according to claim 29, wherein the acidic liquid medium is a solution of an acidic substance in a solvent, more preferably a solution of an acidic substance in water, and wherein the acidic substance is preferably an inorganic acidic substance, such as HCl, H₂SO₂, H₂SO₃, HNO₃, H₃PO₄, H₃PO₃, or H₃PO₂ or a mixture thereof, or the acidic substance is preferably an organic acidic substance, more preferably a carboxylic acid, such as acetic acid or formic acid.

31. A mixture of hydrocarbons obtainable by the method according to claim 16.

32. Use of the mixture of hydrocarbons according to claim 31 for producing chemicals and/or polymers, such as polypropylene and/or polyethylene.
